(19) United States
(12) Patent Application Publication
(10) Pub. No.: US 2020/0323780 A1
(43) Pub. Date: Oct. 15, 2020

(54) BILAYER COMBINATION TABLET FOR ORAL ADMINISTRATION CONTAINING TRAMADOL AND CELECOXIB

(71) Applicants: KIMS HEALTHCARE CO., LTD., Seoul (KR); INDUSTRY-ACADEMIC COOPERATION FOUNDATION, YONSEI UNIVERSITY, Seoul (KR)

(72) Inventors: Seung Hyun KIM, Seoul (KR); Sung Joo HWANG, Seoul (KR); Jun Hyun AHN, Seoul (KR); Sang Min HYUN, Seogwipo-si (KR); Sang Won KWAK, Seogwipo-si (KR); Bo Kyung SUN, Incheon (KR); Seung Hyeon HONG, Danyang-gun (KR); Ye Ji SEO, Cheonan-si (KR)

(73) Assignees: Kims HealthCare Co., Ltd., Seoul (KR); Industry-Academic Cooperation Foundation, Yonsei University, Seoul (KR)

(21) Appl. No.: 16/500,311

(22) PCT Filed: Apr. 3, 2018
(86) PCT No.: PCT/KR2018/003905

§ 371 Date: Oct. 2, 2019

(30) Foreign Application Priority Data
Apr. 3, 2017 (KR) 10-2017-0042927
Apr. 3, 2018 (KR) 10-2018-0038579

(51) Int. Cl. A61K 9/24; A61K 31/137; A61K 31/415; A61K 47/38; A61K 47/34

(52) U.S. Cl. CPC A61K 9/209; A61K 31/137; A61K 47/34; A61K 47/38; A61K 31/415

(57) ABSTRACT

A bilayer combination tablet for oral administration, which is prescribed for alleviation of symptoms or signs of osteoarthritis (degenerative arthritis), alleviation of symptoms or sings of rheumatoid arthritis, alleviation of symptoms or signs of ankylosing spondylitis, alleviation of acute pain in adults (post-operative or post-tooth extraction pain), alleviation of primary dysmenorrhea, or alleviation of severe and moderate acute or chronic pain. The bilayer combination tablet includes: a controlled-release layer containing tramadol as an active ingredient; and an immediate-release layer containing celecoxib as an active ingredient. A composition for treating pain may be provided, which has a long-lasting analgesic effect for 12 hours while reducing the side effects of tramadol by slow release of water-soluble tramadol and immediate release of poorly soluble celecoxib. In addition, the two drugs may be formulated into a single dosage form, and thus the number of medications may be reduced, thereby improving the convenience of drug administration.

### TECHNICAL FIELD

The present disclosure relates to a bilayer combination tablet for oral administration containing tramadol and celecoxib, and more particularly to a bilayer combination tablet for oral administration that maintains a constant dosage interval while exhibiting independent release properties of an immediate release layer and a controlled release layer.

### BACKGROUND ART

Celecoxib is a very poorly water-soluble drug that has a solubility of 3.3 mg/L in water solubility at 20° C. In particular, it has a long in vivo half-life (about 11 hours), and thus the medicinal effect thereof is lasting for about 12 hours after single-dose administration. On the other hand, tramadol is very soluble in water and its blood half-life is short (about 6 hours), and hence the duration of its medicinal effect is also very short. Therefore, formulating these two drugs into a combination tablet is significantly difficult due to the difference in release rate between the two drugs. In particular, when a combination formulation of the two drugs is prepared by simple combination without considering the release rates at all, there is a high risk of not achieving sufficient drug effects as expected.

In recent years, the cases of side effects of tramadol have been reported frequently in the clinical field, and for this reason, some have asserted that it is necessary to change requirements for the approval of tramadol. According to the Health Korea News article “anti-inflammatory analgesic ‘Ultracet’ is dangerous.”, in the United States, there are established standards of the patient's age and duration of administration for the usual dose of tramadol, but in Korea, there is no restriction on the usual dose of tramadol. In addition, according to the article “drugs with the most side effects” of Korea Pharmaceutical Association News (2011), it can be seen that among the top 30 items in terms of side effects reported over five years, Tridol Injection ranks the first and Ultracet Tablet ranks the fifth, and thus single or combination formulations containing tramadol frequently show side effects. Therefore, in Korea, due to the side-effect problems that can occur during long-term administration of a single or combination formulation containing tramadol, it is required to develop a formulation exhibiting a long-lasting analgesic effect while reducing the dose of tramadol to the range that shows no side effects. The development of this formulation is more necessary for patients who need to take tramadol over a long period of time in order to treat pain.

The present disclosure relates to a bilayer combination tablet that reduces the side effects of tramadol (nausea, vomiting, constipation, dizziness, respiratory anxiety, etc.) by reducing the dose of tramadol compared to the usual dose and slowing the release of tramadol in a controlled manner, and at the same time, compensates for the reduced analgesic effect by immediately releasing celecoxib, which exhibits a long-lasting analgesic effect and has relatively less side effects.

### DISCLOSURE

### Technical Problem

The present disclosure has been made in order to solve the above-described problems, and it is an object of the present disclosure to provide a combination anti-inflammatory analgesic that adjusts the effect duration of tramadol similar to the effect duration of celecoxib by slowing down the release rate of tramadol and at the same time, reduces the side effects of tramadol by reducing the dose of tramadol, and to provide a combination formulation for treating acute/chronic pain, and also to improve the convenience of drug administration by formulating the two drugs into a single dosage form.

### Technical Solution

The present disclosure has been made in order to achieve the above-described object, and provides a bilayer combination tablet for oral administration including: a controlled-release layer containing tramadol as an active ingredient; and an immediate-release layer containing celecoxib as an active ingredient, wherein the content ratio of tramadol to celecoxib is 1:4.5 to 1:10.5 by molar ratio.

Moreover, the combination tablet may improve medication compliance so as to enable it to be taken twice a day, by releasing tramadol and celecoxib at different rates such that tramadol is released slowly and celecoxib is released immediately.

Furthermore, the content ratio of tramadol to celecoxib may be 1:5 to 1:13.5 by weight ratio.

In addition, the controlled-release layer may include, as a drug release-controlling agent for controlling the release rate of tramadol, a cellulose derivative, polyether, polyacrylic acid, polyvinyl alcohol, or a mixture of two or more thereof.

In addition, the controlled-release layer include, as a drug release-controlling agent for controlling the release rate of tramadol, one or more selected from the group consisting of cellulose derivatives, including hydroxyethyl cellulose, hydroxypropyl cellulose, carboxymethyl cellulose, and hydroxypropyl methylcellulose; polyethers, including polyethylene oxide and polypropylene oxide; polyacrylic acids, including carbomer; and polyvinyl alcohol.

In addition, the drug release-controlling agent may include hydroxypropyl methylcellulose and polyethylene oxide in amounts of 20 to 30 parts by weight and 30 to 40 parts by weight, respectively, based on 100 parts by weight of the controlled-release layer.

In addition, the content of tramadol in the controlled-release layer may be such that a single dose of tramadol is 35 mg or less.

In addition, the content of tramadol in the controlled-release layer may be such that the single dose of tramadol is 25 mg and the daily dose of tramadol is 50 mg.

### Advantageous Effects

According to the present disclosure, a composition for treating pain may be provided, which has a long-lasting analgesic effect for 12 hours while reducing the side effects of tramadol by slow release of water-soluble tramadol and immediate release of poorly water-soluble celecoxib. In addition, the two drugs may be formulated into a single dosage form, and thus the number of medications may be reduced, thereby improving the convenience of drug administration.

In addition, it is possible to provide a formulation suitable for patients, such as rheumatoid arthritis and spondylitis patients, who take analgesic anti-inflammatory drugs for a long period of time.

### DESCRIPTION OF DRAWINGS

FIG. 1 illustrates a side view and a plan view of a bilayer combination tablet of tramadol and celecoxib according to an embodiment of the present disclosure.

FIGS. 2A and 2B are graphs showing the dissolution rate of celecoxib in Comparative Example 1 and celecoxib single-layer tablets of Examples 1 to 6 of the present disclosure.

FIGS. 3A and 3B are graphs showing the dissolution rate of tramadol in Comparative Example 2 and tramadol single-layer tablets of Examples 7 to 12 of the present disclosure.

FIG. 4A is a graph showing the dissolution rate of a celecoxib immediate-release layer in each of bilayer combination tablets of Examples 13 to 16 of the present disclosure.

FIG. 4B is a graph showing the dissolution rate of a tramadol controlled-release layer in each of bilayer combination tablets of Examples 13 to 16 of the present disclosure.

FIGS. 5 and 6 show images of rat's hind paws before and after the development of rheumatoid arthritis, respectively.

FIG. 7 is a graph showing the results of analyzing Test Example 1 according to the present disclosure and Comparative Examples 1 to 3 by a Mann-Whitney U-test.

FIG. 8 is a perspective view of conditioned place preference boxes which are used to perform a test for measuring psychological dependence which is one of the side effects of tramadol.

FIG. 9 is a graph showing the results of the test for measuring psychological dependence according to an embodiment of the present disclosure.

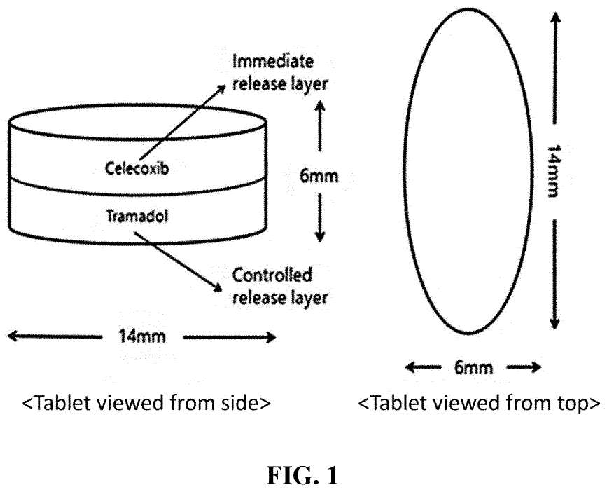

### BEST MODE

The present disclosure is directed to a bilayer combination tablet for oral administration including: a controlled-release layer containing tramadol as an active ingredient; and an immediate-release layer containing celecoxib as an active ingredient, wherein the content ratio of tramadol to celecoxib is 1:4.5 to 1:10.5 by molar ratio.

Hereinafter, the present disclosure will be described in detail with reference to the accompanying drawings.

The present disclosure uses tramadol and celecoxib as active ingredients to prepare a medicament for the treatment of pain, and the two drugs greatly differ in their dissolution rate in the human body. Thus, in order to effectively exhibit the effects of the drugs, mutual control of the release rates of the drugs is necessary. Therefore, the tablet is divided with a layer, which slowly releases tramadol having good solubility in water, and a layer which immediately releases poorly water-soluble celecoxib, so that it may exhibit a lost-lasting analgesic effect for 12 hours. In this case, medication compliance may be improved so that the tablet may be taken twice a day.

In addition, the content of tramadol in the tramadol controlled-release layer of the tablet composition is controlled such that it does not exceed 35 mg per tablet in consideration of the side effects of the active ingredient tramadol.

In addition, for long-lasting drug release effects, the content ratio of tramadol to celecoxib is preferably 1:4.5 to 1:10.5 by molar ratio, and is preferably 1:5 to 1:13.5 by weight.

Furthermore, in an embodiment according to the present disclosure, for long-lasting drug release effects, the content ratio of tramadol to celecoxib is more preferably 1:5.2 to 1:10.5 by molar ratio, and is more preferably 1:6.6 to 1:13.5 by weight ratio.

A binder that is used in the celecoxib immediate-release layer of the present disclosure may include one or more selected from among povidone, copovidone, methyl cellulose, hydroxymethyl cellulose, hydroxypropyl methylcellulose, hydroxypropyl cellulose, hydroxyethyl cellulose, gelatin, guar gum, and xanthan gum. A disintegrant that is used in the celecoxib immediate-release layer of the present disclosure may include one or more selected from among crospovidone, starch, sodium glycolate, croscarmellose sodium, L-hydroxypropyl cellulose, calcium carboxymethyl cellulose, grain starch, alginic acid, sodium alginate, and guar gum. An excipient that is used in the celecoxib immediate-release layer of the present disclosure may include one or more selected from among lactose, dextrose, sucrose, dextrate, mannitol, sorbitol, xylitol, sodium chloride, potassium chloride, magnesium chloride, calcium hydrogen phosphate, calcium phosphate, citric acid, and microcrystalline cellulose. A lubricant that is used in the celecoxib immediate-release layer of the present disclosure may include one or more selected from among colloidal silica, magnesium trisilicate, starch, talc, tricalcium phosphate, magnesium stearate, aluminum stearate, calcium stearate, stearic acid, sodium stearyl fumarate, magnesium carbonate, and magnesium oxide.

Specifically, the celecoxib immediate-release layer of the present disclosure preferably includes, as a main component, celecoxib; as a binder, polyvinylpyrrolidone; as a disintegrant, croscarmellose sodium; as an excipient for direct compression, microcrystalline cellulose; as a surfactant, sodium lauryl sulfate; and as a lubricant, aerosil and magnesium stearate.

Regarding the content of each component, the celecoxib immediate-release layer may include 60.0 to 70.0 wt % of celecoxib, 1.0 to 4.0 wt % of polyvinylpyrrolidone, 4.0 to 6.0 wt % of croscarmellose sodium, 20.0 to 30.0 wt % of microcrystalline cellulose, 0.5 to 1.0 wt % of sodium lauryl sulfate, 0.1 to 0.5 wt % of aerosil, and 0.7 to 1.0 wt % of magnesium stearate.

In addition, the composition of the celecoxib immediate-release layer preferably includes 66.7 wt % of celecoxib, 3.3 wt % of polyvinylpyrrolidone, 5 wt % of croscarmellose sodium, 20.0 to 20.7 wt % of microcrystalline cellulose, 3.3 wt % of sodium lauryl sulfate, 0.3 wt % of aerosol, and 0.7 wt % of magnesium stearate.

An excipient that is used in the tramadol controlled-release layer of the present disclosure may include one or more selected from among lactose, dextrose, sucrose, dextrate, mannitol, sorbitol, xylitol, sodium chloride, potassium chloride, magnesium chloride, calcium hydrogen phosphate, calcium phosphate, citric acid, and microcrystalline cellulose. A drug release-controlling agent that is used in the tramadol controlled-release layer of the present disclosure may include one or more water-soluble polymers selected from among cellulose derivatives (hydroxyethyl cellulose, hydroxypropyl cellulose, carboxymethyl cellulose, and hydroxypropyl methylcellulose), polyethers (polyethylene oxide, and polypropylene oxide), polyacrylic acids (carbomer), and polyvinyl alcohols. A lubricant that is used in the tramadol controlled-release layer of the present disclosure may include one or more selected from among colloidal silica, magnesium trisilicate, starch, talc, tricalcium phosphate, magnesium stearate, aluminum stearate, calcium stearate, stearic acid, sodium stearyl fumarate, magnesium carbonate, and magnesium oxide.

Specifically, a composition of the tramadol controlled-release layer of the present disclosure includes, as a main component, tramadol; as a filler, pre-gelatinized starch; as a drug release-controlling agent, hydroxypropyl methylcellulose and polyethylene oxide; and as a lubricant, aerosil and magnesium stearate.

In addition, the composition of the tramadol controlled-release layer of the present disclosure may include 6.0 to 12.0 wt % of tramadol, 10.0 to 30.0 wt % of pre-gelatinized starch, 20.0 to 30.0 wt % of hydroxypropyl methylcellulose, 30.0 to 40.0 wt % of polyethylene oxide, 1 to 2.5 wt % of aerosil, and 1 to 5 wt % of magnesium stearate.

In addition, the composition of the tramadol controlled-release layer of the present disclosure preferably includes 10.0 wt % of tramadol, 16.0 wt % of pre-gelatinized starch, 32.0 wt % of hydroxypropyl methylcellulose, 36.0 wt % of polyethylene oxide, 2.0 wt % of aerosil, and 4.0 wt % of magnesium stearate.

One embodiment of the present disclosure is directed to a method for preparing a bilayer combination formulation for oral administration containing tramadol and celecoxib as active ingredients. According to this method, in order to reduce the side effects of tramadol and obtain a long-lasting analgesic effect, the bilayer combination formulation is prepared so that tramadol may be released slowly and celecoxib may be released immediately. In this case, the drugs may exhibit a long-lasting analgesic effect in combination for 12 hours, and thus side effects that may occur during long-term administration of analgesics may be reduced, and medication compliance may be improved by combining the two drugs.

The present disclosure is directed to a method for preparing a combination formulation for pain treatment, including the following process steps:

(a) Step of Preparing a Tramadol Controlled-Release Layer

Tramadol, starch 1500, hydroxypropyl cellulose 2208, and polyethylene oxide N12K are mixed with one another, and then ethanol as a binder is added thereto. At this time, ethanol is not completely kneaded with the powder, but is lightly sprayed on the powder, and ethanol and the powder are blended with each other. Next, the blend is sieved through a No. 20 sieve, and then dried in an oven at 60° C. for 6 hours and sieved again through a No. 25 sieve. Aerosil (colloidal silicon dioxide) and magnesium stearate are added to and mixed with the sieved granules, and then granules for a tramadol controlled-release layer are fmally obtained.

(b) Step of Preparing a Celecoxib Immediate-Release Layer

Celecoxib, croscarmellose sodium and microcrystalline cellulose are mixed with one another, and then an ethanol solution containing the binder Povidone K30 and sodium lauryl sulfate dissolved therein is added to and kneaded with the mixture. Next, the kneaded mixture is sieved through a No. 20 sieve, and then dried in an oven at 60° C. for 6 hours and sieved again through a No. 25 sieve. After the sieving, microcrystalline cellulose, Aerosil (colloidal silicon dioxide) and magnesium stearate are added to and mixed with the sieved granules, and then granules for a celecoxib immediate-release layer are fmally obtained.

(c) Step of filling a die with the tramadol layer granules of (a) to establish a tramadol layer, adding the celecoxib layer of (b) onto the tramadol layer, and producing a bilayer combination tablet through complete compression.

Hereinafter, the present disclosure will be described in more detail with reference to examples. These examples are only to illustrate the present disclosure in more detail, and it will be obvious to those skilled in the art that the scope of the present disclosure as defined in the claims of the present disclosure is not limited by these examples.

### MODE FOR INVENTION

### Examples

The compositions of a celecoxib single layer, a tramadol single layer and a bilayer combination tablet are summarized in Tables 1, 2 and 3, respectively.

### Comparative Example 1: A Celecoxib Single Formulation (Celebrex 200 mg Capsule, Pfizer Pharmaceuticals Inc.)

### Comparative Example 2: A Tramadol Single Formulation (Tridol Controlled-Release Tablet 100 Mg, Yuhan Corp.)

### Example 1 to Example 6

Table 1 below shows the compositions of celecoxib single-layer formulations of Examples 1 to 6.

TABLE 1

| | | Weight per tablet (mg) | | | | | |
|---|---|---|---|---|---|---|---|
| Processes | Components | Example 1 | Example 2 | Example 3 | Example 4 | Example 5 | Example 6 |
| Pre-mixing | Celecoxib | 200 | 200 | 200 | 200 | 200 | 200 |
| | Povidone K30 | 15 | | | | 8 | |
| | Croscarmellose | 15 | 15 | | 15 | 15 | 15 |
| | sodium (Acdisol) | | | | | | |
| | Microcrystalline | 100 | 50 | 50 | | | 50 |
| | cellulose (Avicel | | | | | | |
| | PH101) | | | | | | |
| | Lactose | | | | 40 | 50 | |
| Kneading | Povidone K30 | 5 | 10 | 10 | 10 | | 10 |
| | Sodium lauryl | 2 | 2 | 2 | 2 | 5 | 2 |
| | sulfate | | | | | | |
| | Solvent | Ethanol | Ethanol | Ethanol | Ethanol | Distilled water | Ethanol |
| | | (0.05 ml) | (0.05 ml) | (0.05 ml) | (0.05 ml) | (0.05 ml) | (0.05 ml) |
| Sieving (20 mesh) | | | | | | | |
| Drying (60° C., dry oven, 6 hours) | | | | | | | |
| Sieving (25 mesh) | | | | | | | |
| Post-mixing | Croscarmellose | | | 15 | | | |
| | sodium (Acdisol) | | | | | | |
| | Microcrystalline | 11 | 20 | 20 | | 20 | 20 |
| | cellulose (Avicel | | | | | | |
| | PH101) | | | | | | |
| | Aerosil (200 | 1 | 1 | 1 | 1 | | 1 |
| | Pharma) | | | | | | |
| | Magnesium | 2 | 20 | 20 | 2 | 2 | 2 |
| | stearate | | | | | | |
| Compression | Sum | 351 | 318 | 318 | 270 | 300 | 300 |
| into tablet | | | | | | | |

### Example 7 to Example 12

Table 2 below shows the compositions of tramadol single-layer formulations of Examples 7 to 12.

TABLE 2

| | | Weight per tablet (mg) | | | | | |
|---|---|---|---|---|---|---|---|
| Processes | Components | Example 7 | Example 8 | Example 9 | Example 10 | Example 11 | Example 12 |
| Pre-mixing | Tramadol | 25 | 25 | 25 | 25 | 25 | 25 |
| | hydrochloride | | | | | | |
| | Starch 1500 | 50 | 50 | 60 | 70 | 40 | 30 |
| | Hydroxypropyl | 70 | 80 | 60 | 50 | 80 | 80 |
| | methylcellulose | | | | | | |
| | 2208 | | | | | | |
| | Polyethylene oxide | 90 | 80 | 90 | 90 | 90 | 100 |
| | N12K | | | | | | |
| Kneading | solvent | Ethanol (0.05 ml) | | | | | |
| Sieving (20 mesh) | | | | | | | |
| Drying (60° C., dry oven, 6 hours) | | | | | | | |
| Sieving (25 mesh) | | | | | | | |
| Post-mixing | Aerosil (200 vv | 5 | 5 | 5 | 5 | 5 | 5 |
| | Pharma) | | | | | | |
| | Magnesium | 10 | 10 | 10 | 10 | 10 | 10 |
| | stearate | | | | | | |
| Compression | Sum | 250 | 250 | 250 | 250 | 250 | 250 |
| into tablet | | | | | | | |

### Example 13 to Example 16

Table 3 below shows the compositions of bilayer formulations of Examples 13 to 16.

TABLE 3

| | | Weight per tablet (mg) | | | |
|---|---|---|---|---|---|
| Processes | Components | Example 13 | Example 14 | Example 15 | Example 16 |

Celecoxib immediate-release layer

| Pre-mixing | Celecoxib | 200 | 200 | 200 | 200 |
|---|---|---|---|---|---|
| | Croscarmellose | 15 | 15 | 15 | 15 |
| | sodium | | | | |
| | (Acdisol) | | | | |
| | Microcrystalline | 50 | 50 | 50 | 50 |
| | cellulose | | | | |
| | (Avicel PH101) | | | | |
| | Lactose | | | | |
| Kneading | Povidone K30 | 10 | 10 | 10 | 10 |
| | Sodium lauryl | 2 | 2 | 2 | 2 |
| | sulfate | | | | |
| | Solvent | 0.05 ml | 0.05 ml | 0.05 ml | 0.05 ml |
| | (ethanol) | | | | |
| Sieving (20 mesh) | | | | | |
| Drying (60° C., dry oven, 6 | | | | | |
| hours) | | | | | |
| Sieving (25 mesh) | | | | | |
| Post-mixing | Microcrystalline | 20 | 20 | 20 | 20 |
| | cellulose (Avicel | | | | |
| | PH101) | | | | |
| | Aerosil (200 | 1 | 1 | 1 | 1 |
| | Pharma) | | | | |
| | Magnesium stearate | 20 | 20 | 2 | 2 |

Tramadol controlled-release layer

| Pre-mixing | Tramadol | 25 | 25 | 25 | 25 |
|---|---|---|---|---|---|
| | hydrochloride | | | | |
| | Starch 1500 | 40 | 30 | 40 | 30 |
| | Hydroxypropyl | 80 | 80 | 80 | 80 |
| | methylcellulose | | | | |
| | 2208 | | | | |
| | Polyethylene oxide | 90 | 100 | 90 | 100 |
| | N12K | | | | |
| Kneading | Solvent (ethanol) | 0.05 ml | 0.05 ml | 0.05 ml | 0.05 ml |
| Sieving (20 mesh) | | | | | |
| Drying (60° C., dry oven, 6 hours) | | | | | |
| Sieving (25 mesh) | | | | | |
| Post-mixing | Aerosil (200 vv | 5 | 5 | 5 | 5 |
| | Pharma) | | | | |
| | Magnesium stearate | 10 | 10 | 10 | 10 |

| | | | | |
|---|---|---|---|---|
| Sum | 568 | 568 | 550 | 550 |

### Test Example

1. Standard and Test Method (Dissolution Test) for Raw Drugs

The apparatus of USP method 2 (paddle method) was used to measure the dissolution rates of the tramadol controlled-release layer, the celecoxib immediate-release layer and Comparative Examples 1 and 2. For tramadol, 900 ml of D.W. was used as a dissolution fluid, and for celecoxib, 900 ml of 1% sodium lauryl sulfate solution was used as a dissolution fluid. The solution was maintained at a temperature of 37±2° C. and stiffed at 50 rpm during the test. Twelve identical tablets or capsules were each separately placed in one of standard dissolution vessels, and a 2 ml aliquot of solution was obtained from each vessel at each time point. The sample from each vessel was filtered through a 0.45 μm PTFE filter, and the concentration was measured through HPLC-UV.

[Test Results]

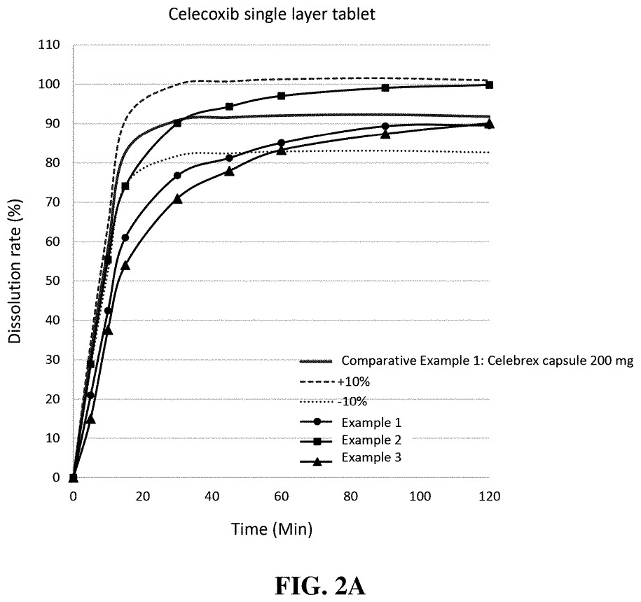

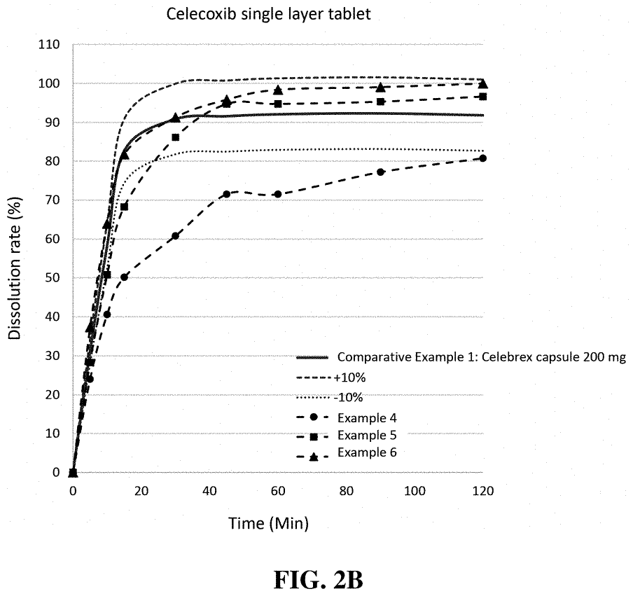

FIGS. 2A and 2B are graphs showing the dissolution rate of celecoxib for Comparative Example 1 and the celecoxib single-layers of Examples 1 to 6. The celecoxib capsule of Comparative Example 1 which is an immediate-release formulation showed an initial dissolution rate of celecoxib of 80% or higher within 15 minutes. Examples 1, 3, 4 and 5 showed a significant difference in the initial dissolution rate from Comparative Example 1, whereas Examples 2 and 6 showed initial dissolution rates similar to that of Comparative Example 1 within 15 minutes. Thus, it was confirmed that Examples 2 and 6 are suitable formulations for a celecoxib immediate-release layer.

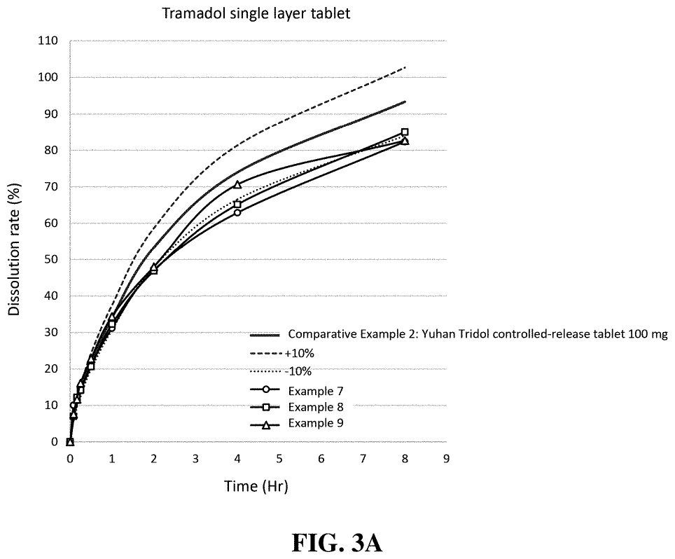

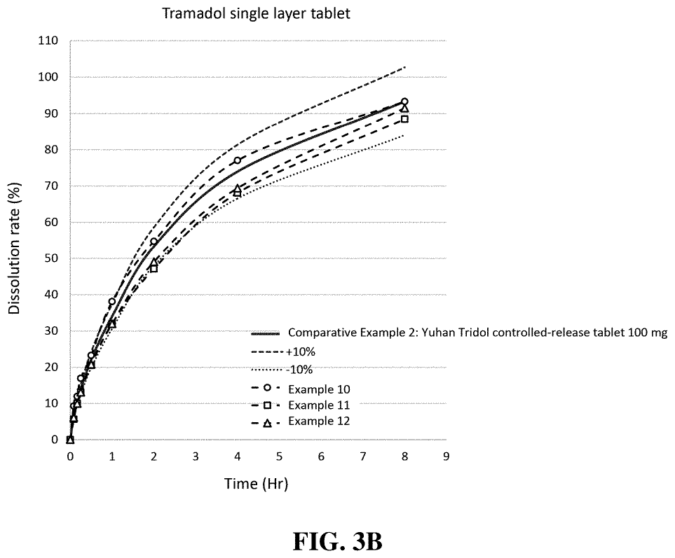

FIGS. 3A and 3B are graphs showing the dissolution rate of tramadol for Comparative Example 2 and the tramadol single-layers of Examples 7 to 12. In the case of the Yuhan Tridol controlled-release tablet of Comparative Example 2 which is a controlled-release formulation, it could be seen that the dissolution rates after 4 hours and 8 hours were 70% and 90%, respectively. It was confirmed that the Examples, except for Examples 7 and 8, did not show a significant difference in the dissolution rate from Comparative Example 2, but in the case of Examples 7 to 10, the dissolution rate of tramadol in the bilayer combination tablets was 70% or higher at 2 hours and 90% or higher at 4 hours, as shown in the previous studies, and thus the control of release of tramadol was not achieved properly. Thus, it was confirmed that Examples 11 and 12 are suitable formulations for a tramadol controlled-release layer.

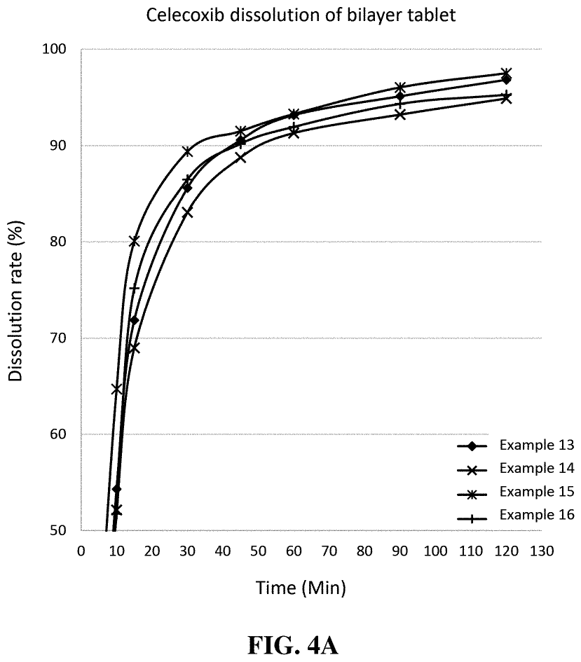

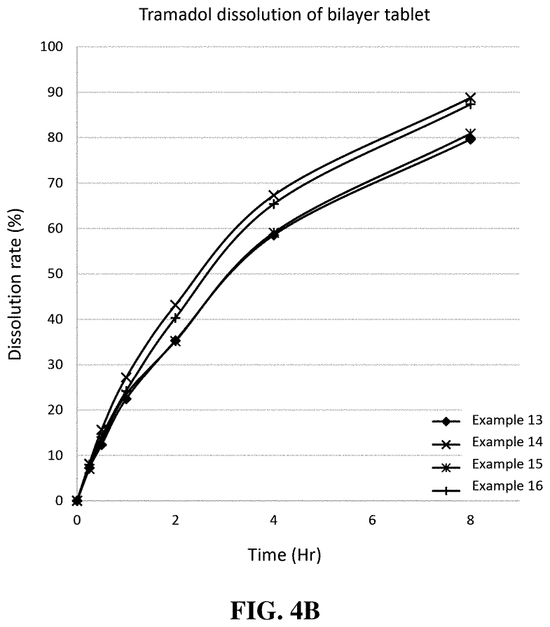

FIG. 4A is a graph showing the dissolution rate of celecoxib in the immediate-release layer in each of the bilayer combination tablets. Examples 13 to 16 all showed a dissolution rate of celecoxib of 90% or higher after 1 hour, but Examples 13 and 14 showed an initial dissolution rate of celecoxib of 80% or higher at 15 minutes. However, in terms of the dissolution rate in the late stage, Example 13 showed a higher dissolution rate than Example 14. Thus, it was confirmed that Example 13 is the best suitable formulation.

FIG. 4B is a graph showing the dissolution rate of tramadol in the controlled-release layer in each of the bilayer combination tablets. It was confirmed that Examples 13 and 15 showed similar dissolution rates, Examples 14 and 16 showed similar dissolution rates, and the dissolution rates of Examples 13 and 15 were lower than those of Examples 14 and 16. In addition, although there was no significant difference in the dissolution rate between Examples 13 and 15, the release of tramadol in Example 13 was better controlled than that in Example 15, indicating that Example 13 is the best suitable formulation.

The above description of the drawings shows the results of dissolution of tramadol and celecoxib in the tramadol single layer, the celecoxib single layer and the bilayer combination tablets in Examples 1 to 16. It could be conformed through Comparative Example 1 that the celecoxib single layer is an immediate-release formulation when it shows an initial dissolution rate of 80% or higher at 15 minutes. In addition, it could be confirmed that Examples 2 and 6 are suitable formulations. In the case of the tramadol single layer, it was confirmed that the Yuhan Tridol controlled-release tablet showed dissolution rates of 70% and 90% at 4 hours and 8 hours, respectively, and it could be confirmed that Examples 11 and 12 are suitable formulations. Based on the results of dissolution of celecoxib and tramadol in the single layers, the dissolution of celecoxib and tramadol in the bilayer combination tablets of Examples 13 to 16 was examined. As a result, it was confirmed that Example 13 is the best suitable formulation for immediate released of celecoxib and slow release of tramadol.

In addition, it can be seen that the immediate-release layer showed a dissolution rate of 80% or higher within 15 minutes, a dissolution rate of 90% within 45 minutes, and a dissolution rate close to 100% within 120 minutes, when tested by the USP II device in 900 ml of 1% SLS (sodium lauryl sulfate) at 37° C. and 50 rpm, and the controlled-release layer showed a dissolution rate of 60% for 4 hours, a dissolution rate of 80% for 8 hours, and a dissolution rate close to 100% for 12 hours, when tested by the USP II device in 900 ml of distilled water at 37° C. and 75 rpm.

2. Test for Measuring Analgesic Effect in Type 2 Collagen-Induced Rheumatoid Arthritis Model

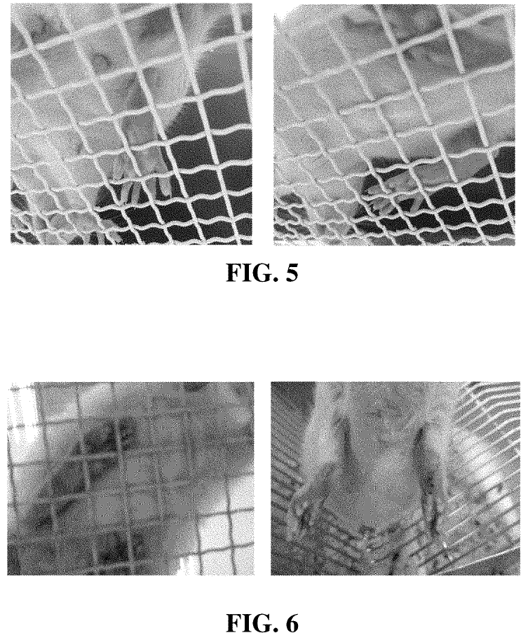

7-Week-old male Sprague-Dawley rats (SD rats) were divided into test groups, and then rheumatoid arthritis was induced in the rats. For induction of rheumatoid arthritis, bovine type-2 collagen was dissolved in 0.05 M acetic acid aqueous solution to prepare 2 mg/ml of a collagen solution, and the collagen solution was mixed with incomplete Freund's adjuvant (IFA). The mixture was first injected intradermally 2 cm below the base of the tail of each SD rat, and after 1 week, the mixture was further injected in the same manner. When rheumatoid arthritis was induced, redness and edema were observed in the rat's hind paws. As can be seen in FIG. 5 (before the development of rheumatoid arthritis) and FIG. 6 (after the development of rheumatoid arthritis), redness and edema could be visually observed.

The pain occurring when rheumatoid arthritis was induced and the analgesic effect after drug treatment were evaluated by measuring paw withdrawal threshold (PWT) using Von-frey filaments. For evaluation of the analgesic effect, Von-frey filaments with different intensities are approached to the paw surface in the order of strength (from lower strength to higher strength), and a mechanical stimulus was applied five times to the paw such that the filament would be slightly bent. When there was no withdrawal response after application of the mechanical stimulus, a stronger filament was applied, and when the paw withdrawal response occurred three times or more, the strength of the corresponding filament was determined and evaluated as paw withdrawal threshold.

Before Test Example 1 and Comparative Examples 1 and 2 were injected intraperitoneally as shown in Table 4 below, PWT was measured using Von-frey filaments, and Test Example 1 and Comparative Examples 1 and 2 were injected intraperitoneally. At 30 minutes after the intraperitoneal injection, PWT was measured in the same manner. Next, the change trend of PWT was calculated using Equation 1 below, thereby evaluating the analgesic effect.

Analgesic effect=PWT after injection−PWT before injection [Equation 1]

Animal equivalent dose (AED) depending on human dose was calculated using Equation 2 below.

$$A E D ( \frac{mg}{kg} ) = Human dose ( \frac{mg}{kg} ) \times ( \frac{Human {K}_{m}}{Animal {K}_{m}} ) \qquad [ Equation 2 ]$$

In order to compare the Test Example with Comparative Examples 1 to 3 on the basis of all the measurement results, analysis was performed using a Kruskal-Wallis test which is a non-parametric statistical method, and the significance level (p-value) was confirmed. In addition, Mann-Whitney U-test was used to analyze to compare Test Example 1 with each of the Comparative Examples and confirm the significance level (p-value). Statistical processing was performed using IBM® SPSS® Statistics version 24.

TABLE 4

| Test Example and | Sample name | Animal equivalent |
|---|---|---|
| Comparative Examples | and dose (mg) | dose (AED, mg/kg) |
| Test Example 1 | Celecoxib 200 mg + | Celecoxib 17.6 mg/kg + |
| | tramadol 25 mg | tramadol 2.2 mg/kg |
| Comparative | Vehicles | — |
| Example 1 | | |
| Comparative | Celecoxib 200 mg + | Celecoxib 17.6 mg/kg + |
| Example 2 | tramadol 37.5 mg | tramadol 3.3 mg/kg |
| Comparative | Celecoxib 200 mg + | Celecoxib 17.6 mg/kg + |
| Example 3 | tramadol 100 mg | tramadol 8.8 mg/kg |

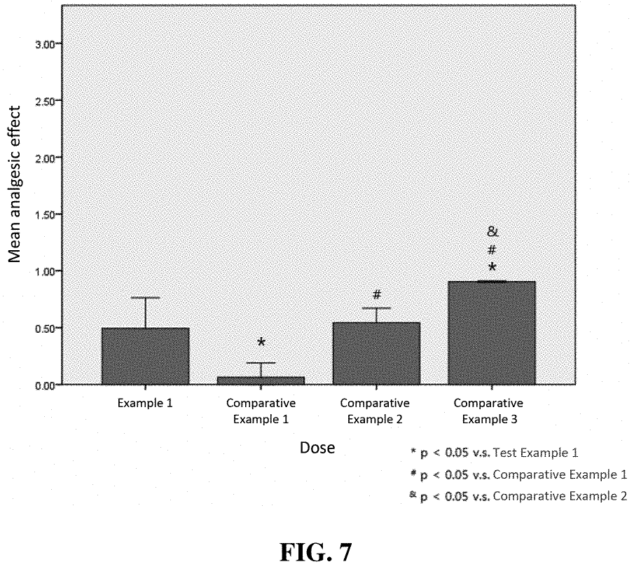

The average value of the analgesic effect of each of Test Example 1 and Comparative Examples 1 to 3 is shown in Table 5 below. Next, based on the population values, the Test Example and the Comparative Examples were analyzed using Kruskal-Wallis test. As a result, the significance level was 0.023 (p<0.05), and there was a statistically significant difference between the groups. Thereafter, each of Test Example 1 and Comparative Examples 1 to 3 were analyzed using Mann-Whitney U-test, and the results of the analysis are shown in FIG. 7. That is, co-administration of celecoxib and tramadol showed an analgesic effect, and the analgesic effect of Test Example 1 was similar to that of Comparative Example 2. In addition, the analgesic effect of Test Example 1 did significantly differ from that of Comparative Example 3.

TABLE 5

| Test Example and Comparative Examples | Analgesic effect |
|---|---|
| Test Example 1 | 0.49 |
| Comparative Example 1 | 0.03 |
| Comparative Example 2 | 0.54 |
| Comparative Example 3 | 0.89 |

3. Test for Measuring Psychological Dependence on Tramadol by Assessment of Dependence Potential

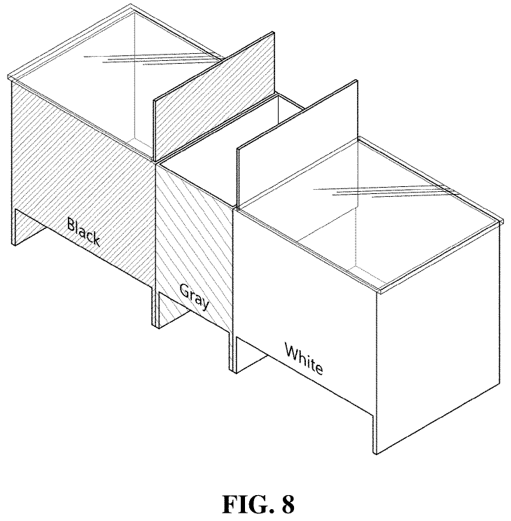

In order to measure psychological dependence which is one of the side effects of tramadol, a change in conditioned place preference (CPP) was measured. Conditioned place preference boxes are divided into three boxes, that is, a white box, a black box and a gray box, as shown in FIG. 8. The gray box is a meddle portion in which an animal can choose between the white box and the black box, and the gray box is 28 cm in length. The black box has a bottom composed of stainless steel rods having a diameter of 4.8 mm and a spacing of 16 mm, and the white box has a bottom composed of stainless steel meshes having a size of 1.25×1.25 cm. The gray box is 12 cm in width, 22 cm in length, and 21 cm in height, and the white box and the black box are 28 cm in width, 21 cm in length, and 21 cm in height.

A series of test procedures are as follows. 5-Week-old male Sprague-Dawley rats (SD rats) were purchased and acclimated for one week. Then, on days 1, 2 and 3 of the test (preconditioning step), in a state in which each rat was not treated with a drug or normal saline for injection, each rat was placed in the gray compartment, and then the door was opened and the time that each rat stayed in each compartment was measured for 15 minutes. Then, based on the mean of the measurement results, the compartment preferred by the rats in the preconditioning step (that is, the compartment in which the rats stayed longer, among the white compartment and the black compartment) was determined. Then, the rats were divided into test groups (Test Example 2 and Comparative Examples 4 to 6) as shown in Table 6 below, and subjected to the next step (conditioning step).

TABLE 6

| Test Example and | Sample name | Animal equivalent |
|---|---|---|
| Comparative Examples | and dose (mg) | dose (AED, mg/kg) |
| Test Example 2 | Tramadol 25 mg | Tramadol 2.6 mg/kg |
| Comparative | Normal saline | — |
| Example 4 | | |
| Comparative | Tramadol 37.5 mg | Tramadol 3.9 mg/kg |
| Example 5 | | |
| Comparative | Tramadol 100 mg | Tramadol 10.3 mg/kg |
| Example 6 | | |

The next step is a conditioning step corresponding to day 4 to day 11. On days 4, 6, 8 and 10, the corresponding dose of each of the Test Example and the Comparative Examples was administered by intraperitoneal injection to each rat, and after 30 minutes, the rats were adapted to the non-preferred compartment for 30 minutes so as to allow the rats to remember the non-preferred compartment in association with tramadol. On the remaining days 5, 7, 9 and 11, normal saline for injection was administered by intraperitoneal injection to the rats, and after 30 minutes, the rats were adapted to the preferred compartment for 30 minutes so as to allow the rats to remember the preferred compartment in association with the saline solution. On day 12 corresponding to a final step (post-conditioning step), in a state in which neither the drug nor normal saline for injection was administered to the rats, the rats were placed in the gray compartment, and then the door was opened and the time that the rats stayed in each compartment was measured for 15 minutes (see Table 7).

TABLE 7

| Days | Step | Treatment | | Adaption | Time |
|---|---|---|---|---|---|
| Day 1 | Pre-conditioning | — | | — | 15 minutes |
| Day 2 | | | | | |
| Day 3 | | | | | |
| Day 4 | Conditioning | Test Example 2 | Tramadol 2.6 | non-preferred | 30 minutes |
| | | | mg/kg | compartment | |
| | | Comparative | Normal saline | | |
| | | Example 4 | | | |
| | | Comparative | Tramadol 3.9 | | |
| | | Example 5 | mg/kg | | |
| | | Comparative | Tramadol 10.3 | | |
| | | Example 6 | mg/kg | | |
| Day 5 | | Test Example 2 | Normal saline | preferred | |
| | | Comparative | | compartment | |
| | | Example 4 | | | |
| | | Comparative | | | |
| | | Example 5 | | | |
| | | Comparative | | | |
| | | Example 6 | | | |
| Day 6 | | Test Example 2 | Tramadol 2.6 | non-preferred | |
| | | | mg/kg | compartment | |
| | | Comparative | Normal saline | | |
| | | Example 4 | | | |
| | | Comparative | Tramadol 3.9 | | |
| | | Example 5 | mg/kg | | |
| | | Comparative | Tramadol 10.3 | | |
| | | Example 6 | mg/kg | | |
| Day 7 | | Test Example 2 | Normal saline | preferred | |
| | | Comparative | | compartment | |
| | | Example 4 | | | |
| | | Comparative | | | |
| | | Example 5 | | | |
| | | Comparative | | | |
| | | Example 6 | | | |
| Day 8 | | Test Example 2 | Tramadol 2.6 | non-preferred | |
| | | | mg/kg | compartment | |
| | | Comparative | Normal saline | | |
| | | Example 4 | | | |
| | | Comparative | Tramadol 3.9 | | |
| | | Example 5 | mg/kg | | |
| | | Comparative | Tramadol 10.3 | | |
| | | Example 6 | mg/kg | | |
| Day 9 | | Test Example 2 | Normal saline | preferred | |
| | | Comparative | | compartment | |
| | | Example 4 | | | |
| | | Comparative | | | |
| | | Example 5 | | | |
| | | Comparative | | | |
| | | Example 6 | | | |
| Day 10 | | Test Example 2 | Tramadol 2.6 | non-preferred | |
| | | | mg/kg | compartment | |
| | | Comparative | Normal saline | | |
| | | Example 4 | | | |
| | | Comparative | Tramadol 3.9 | | |
| | | Example 5 | mg/kg | | |
| | | Comparative | Tramadol 10.3 | | |
| | | Example 6 | mg/kg | | |
| Day 11 | | Test Example 2 | Normal saline | preferred | |
| | | Comparative | | compartment | |
| | | Example 4 | | | |
| | | Comparative | | | |
| | | Example 5 | | | |
| | | Comparative | | | |
| | | Example 6 | | | |

| | | | |
|---|---|---|---|
| Day 12 | Post- | — | 15 minutes |
| | conditioning | | |

The difference between the time that the rats stayed in the tramadol-associated place (non-preferred compartment) in the post-conditioning step and the time that the rats stayed in the non-preferred compartment in the pre-conditioning step indicates the tramadol-induced change in conditioned place preference, that is, psychological dependence on the drug. Thus, a value for this difference was calculated, and the results of the calculation are shown in Table 8 below.

Next, based on the test results, the standardized J-T statistic and the significance level (p-value) were confirmed using a Jonckheere-terpstra test which is a non-parametric statistical method.

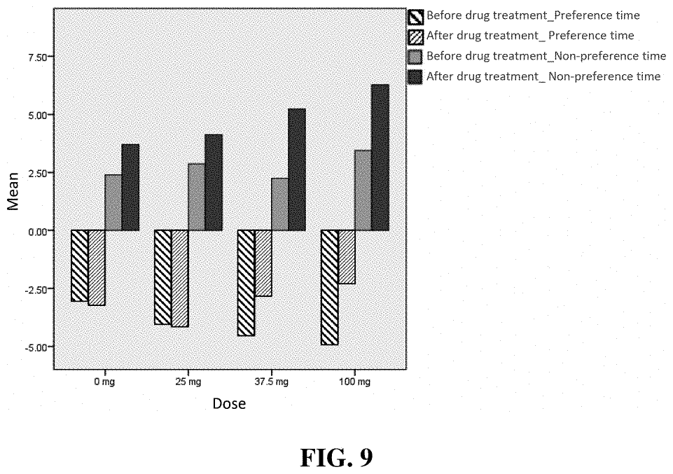

Test Example 2 and Comparative Examples 4 to 6 were analyzed using Jonckheere-terpstra test, and as a result, it could be seen that the standardized J-T statistic was 2.345 and the significance level was 0.019 (p<0.05), indicating that as the concentration of tramadol increased, the time stayed in the non-preferred compartment in the post-conditioning step significantly increased. In addition, it could be seen that as can be seen in FIG. 9, the psychological dependence on tramadol greatly increased in Comparative Example 5 (tramadol 37.5 mg) and Comparative Example 6 (tramadol 100 mg) compared to Test Example 2 (tramadol 25 mg) and Comparative Example 4 (tramadol 0 mg). Such results demonstrate that the psychological dependence on a tramadol dose of 25 mg lower than the psychological dependence on tramadol doses of 37.5 mg and 100 mg.

TABLE 8

| Test Example and | Change (min) in the time | Change (min) in the time |
|---|---|---|
| Comparative | stayed in preferred | stayed in non-preferred |
| Examples | compartment | compartment |
| Test Example 2 | −0.17 | 1.3 |
| Comparative | 0.5 | 1.4 |
| Example 4 | | |
| Comparative | 2.20 | 2.8 |
| Example 5 | | |
| Comparative | 3.03 | 3.3 |
| Example 6 | | |

Therefore, it is confirmed that the bilayer combination tablet for oral administration containing 25 mg tramadol and 200 mg celecoxib according to the present disclosure, which improves medication compliance so as to enable the tablet to be taken twice a day, exhibits an analgesic effect while having less side effects showing psychological dependence, and in particular, the analgesic effect thereof is not inferior to that of a conventional product having a tramadol dose of 37.5 mg or 100 mg.

1. A bilayer combination tablet for oral administration comprising:
a controlled-release layer containing tramadol as an active ingredient; and
an immediate-release layer containing celecoxib as an active ingredient,
wherein a content ratio of tramadol to celecoxib is 1:4.5 to 1:10.5 by molar ratio.

2. The bilayer combination tablet for oral administration of claim 1, wherein the combination tablet improves medication compliance so as to enable it to be taken twice a day, by releasing tramadol and celecoxib at different rates such that tramadol is released slowly and celecoxib is released immediately.

3. The bilayer combination tablet for oral administration of claim 1, wherein the content ratio of tramadol to celecoxib is 1:5 to 1:13.5 by weight ratio.

4. The bilayer combination tablet for oral administration of claim 1, wherein the controlled-release layer comprises, as a drug release-controlling agent for controlling the release rate of tramadol, a cellulose derivative, polyether, polyacrylic acid, polyvinyl alcohol, or a mixture of two or more thereof.

5. The bilayer combination tablet for oral administration of claim 4, wherein the controlled-release layer comprises, as the drug release-controlling agent for controlling the release rate of tramadol, one or more selected from the group consisting of cellulose derivatives, including hydroxyethyl cellulose, hydroxypropyl cellulose, carboxymethyl cellulose, and hydroxypropyl methylcellulose; polyethers, including polyethylene oxide and polypropylene oxide; polyacrylic acids, including carbomer; and polyvinyl alcohol.

6. The bilayer combination tablet for oral administration of claim 5, wherein the drug release-controlling agent comprises hydroxypropyl methylcellulose and polyethylene oxide in amounts of 20 to 30 parts by weight and 30 to 40 parts by weight, respectively, based on 100 parts by weight of the controlled-release layer.

7. The bilayer combination tablet for oral administration of claim 1, wherein the content of tramadol in the controlled-release layer is such that a single dose of tramadol is 35 mg or less.

8. The bilayer combination tablet for oral administration of claim 7, wherein the content of tramadol in the controlled-release layer is such that the single dose of tramadol is 25 mg and a daily dose of tramadol is 50 mg.
